(19) United States
(12) Patent Application Publication
(10) Pub. No.: US 2003/0073608 A1
(43) Pub. Date: Apr. 17, 2003

(54) Process for treating disease

(76) Inventors: Nancy J. Woolf, Studio City, CA (US); Howard J. Greenwald, Penfield, NY (US)

Correspondence Address: GREENWALD & BASCH, LLP, 349 WEST COMMERCIAL STREET, SUITE 2490, EAST ROCHESTER, NY, 14445, US

(21) Appl. No.: 09/832,015

(22) Filed: Apr. 10, 2001

(51) Int. Cl. A61K 31/00; A61K 9/22

(52) U.S. Cl. 514/1; 424/468; 424/423

(57) ABSTRACT

A process for treating Alheimer's disease, comprising the steps of administering to a human patient an antagonist of a neurotransmitter receptor which indirectly inhibits phosporylation of microtubule-associated protein-2, and thereafter administering to said patient and anticholinesterase agent. The antagonist of the neurotransmitter binds to a neurotransmitter receptor which phosphorylates said microtubule-associated protein-2 in limbic cells, the antagonist of the neurotransmitter binds to a neurotransmitter receptor which phosphorylates microtuble-associated protein-2 in neocortical cells, and the antagonist binds to said neurotransmitter receptor in said limbic cells at least 1.5 times as much as it binds to said neurotransmitter receptor in said neocortical cells.

### FIELD OF THE INVENTION

A process for treating neurodegenerative disease in which an antagonist of a neurotransmitter receptor is first administered to a patient, wherein said antagonist indirectly inhibits phosphorylation of microtubule-associated protein-2; and, thereafter, an anticholinesterase agent is then administered to the patient.

### BACKGROUND OF THE INVENTION

Neurodegenerative disease in the United States has reached startling levels, due, at least in part, to the aging of the population. As is known, neurodegenerative disease involves widespread neuronal insult, such as the accumulation of neurofibrillary tangles and senile plaques. Typical neurodegenerative diseases include, e.g., Parkinson's disease-related dementia and Alzheimer's disease.

U.S. Pat. No. 6,937,352 teaches that “Alzheimer's disease and other cognitive disorders have received much attention lately, yet treatments for this disease have not been very successful. According to Melchiorre et al. (J. Med Chem (1993), 36, 3734-3737), compounds that selectively antagonize M2 muscarinic receptors, especially in relation to M1 muscarinic receptors, should possess activity against cognitive disorders.” The entire disclosure of U.S. Pat. No. 6,937,352 is hereby incorporated by reference into this specification.

A similar disclosure is presented in U.S. Pat. No. 5,877,183, the entire disclosure of U.S. Pat. No. 5,877,173 also is incorporated by reference into this specification. This '173 patent provides an excellent and detailed description, in its Background section, on “(1) neurotransmitter receptors in the brain; (2) mechanisms by which these transmitter and receptor systems may contribute to neuronal degeneration in Alzheimer's disease; and (3) certain types of drugs that can be used to prevent or reduce neuronal degeneration in patient's suffering from Alzheimer's disease.” The patent also discusses the difficulty encountered by prior art researchers in combating Alzheimer's disease, stating that: “The extremely complex (and apparently contradictory and paradoxical) neurological systems and processes involved in Alzheimer's disease have stubbornly confounded the efforts of literally thousands of highly skilled researchers and physicians, for decades.”

Similarly, in the July, 2000 edition of newsletter entitled “Neurology Alert” (published by the American Health Consultants, Atlanta, Ga., Volume 18, Number 11, at pages 81-88), it is disclosed that: “Few consultations in neurology are more dispiriting than conveying a diagnosis of Alzheimer's disease . . . to patient and family. Providing this diagnosis is accompanied by the knowledge of an impending, inexorable decline in the patient's most human qualities, with little or no hope for meaningful therapeutic intervention. . . . Indeed, no drugs proven to significantly retard the progression of AD are currently available.”

It is an object of this invention to provide a process for significantly retarding the progression of Alzheimer's disease.

It is another object of this invention to provide a time-release drug-delivery device for practicing the process of this invention.

It is yet another object of this invention to provide an implantable assembly for practicing the process of this invention.

### SUMMARY OF THE INVENTION

In accordance with this invention, there is provided a process for reducing progressive neuronal degeneration due to Alzheimer's disease. In the first step of this process, there is administered to a human patient suffering from or at elevated risk for Alzheimer's disease an antagonist of a neurotransmitter receptor, wherein said antagonist indirectly inhibits phosphorylation of microtubule-associated protein-2. Thereafter, in the second step of this process, an anticholineresterase agent is administered to the patient.

### BRIEF DESCRIPTION OF THE DRAWINGS

The invention will be described by reference to the specification and the drawings, in which like numerals refer to like elements, and wherein:

FIG. 1 is a flow diagram of a process for identifying the preferred antagonists used in the process of the invention, and

FIG. 2 is a schematic representation of another preferred process of the invention.

### DESCRIPTION OF THE PREFERRED EMBODIMENTS

In the process of this invention, an antagonist of a neurotransmitter receptor is first administered to a patient. This antagonist indirectly inhibits phosphorylation of microtubule-associated protein 2.

As is known to those skilled in the art, the MAP-2 microtubule associated protein consists of a pair of high molecular mass (280 kiloDaltons) proteins, MAP-2a/b, and a low molecular mass (70 kiloDaltons) polypeptide, MAP-2c, which are generated by alternative splicing of a single gene. MAP-2 is extensively phosphorylated, and the phosphorylation state of MAP-2 modulates its function and metabolism. See, e.g., an article by N. J. Woolf, M.D. Zinnerman, and G. V. W. Johnson entitled “Hippocampal microtubule-associated protein-2 alterations with contextual memory,” published in Brain Research 821 (1999) at pages 241-249. Reference also may be had, e.g., to U.S. Pat. Nos. 6,200,768, 6,146,827, 6,030,822, 6,020,142, 6,010,913, 5,994,304, 5,989,907, 5,962,424, 5,914,261, 5,872,006, 5,861,257, 5,846,780, 5,795,735, 5,776,751, 5,767,252, 5,753,505, 5,595,904, 5,580,898, 5,459,036, 5,385,915, and the like. The disclosure of each of these United States patents is hereby incorporated by reference into this specification.

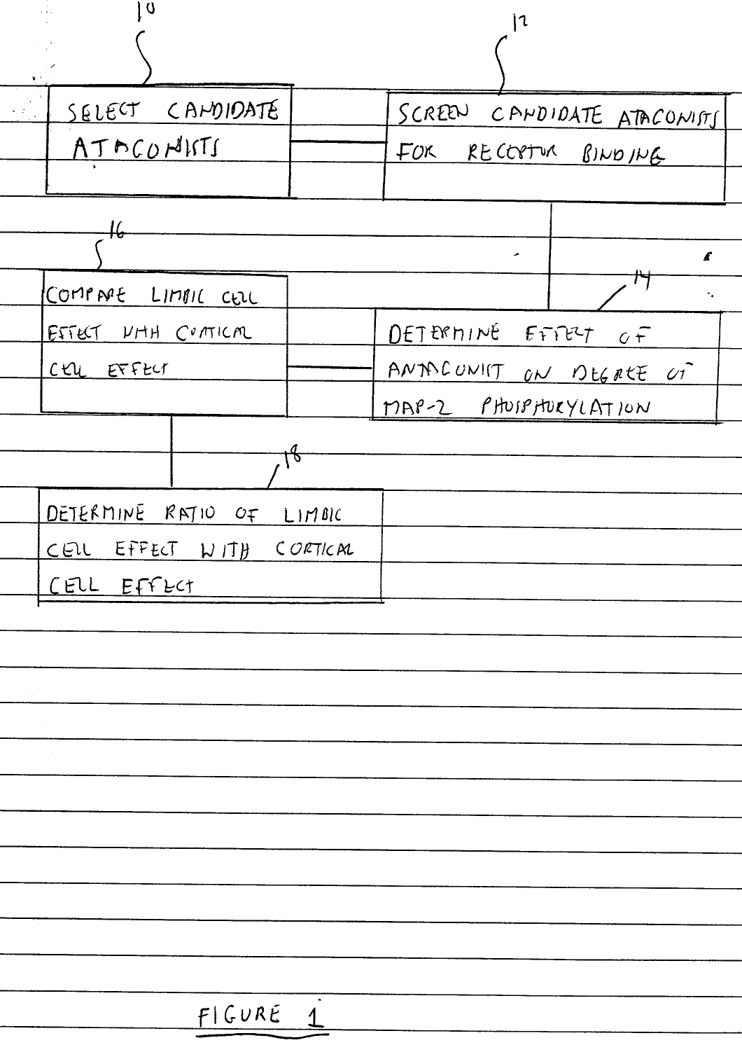

The MAP-2 antagonist used in the process of this invention may be selected by the process described in FIG. 1. In step 10 of such process, some candidate antagonists which might work in the process of the invention are first selected to be screened. The candidate antagonists should have properties such that they bind to a membrane receptor that acts on a second messenger that, in turn, acts on a protein kinase adapted to phosphorylate MAP-2. Furthermore, the candidate antagonists should preferably selectively antagonize MAP-2 in the limbic area.

By way of illustration, muscarinic antagonists are suitable candidate antagonists for the selection process depicted in FIG. 1. Muscarinic antagonists block the actions of muscarinic receptors. Muscarinic receptors are synaptic acetylcholine receptors to which muscarine binds, thereby mimicking the action of acetylcholine. These receptors are found at smooth muscle end plates and in the brain. See, e.g., U.S. Pat. Nos. 6,171,780, 6,159,705, 6,127,133, 6,093,733 (muscarinic receptor agonists), 6,093,545 (methods for detecting nucleic acid molecules encoding a member of the muscarinic family of receptors), 6,057,114, 6,017,734, 6,010,861, 5,958,919, 5,955,281, 5,945,307, 5,912,132, 5,882,898, 5,880,129, 5,877,199, 5,877,173, 5,852,014, 5,747,336, 5,739,119, 5,726,179, 5,714,666, 5,707,798, 5,705,514, 5,670,113, 5,605,911, 5,498,620, 5,446,057, 5,436,128, 5,407,938, 5,403,845, 5,401,629, 5,384,243, 5,359,078, 5,324,832, 5,175,166, and the like. The entire disclosure of each of these United States patents is hereby incorporated by reference into this specification.

Muscarinic antagonists are also well known to those skilled in the art. Thus, by way of illustration and not limitation, these muscaranic antagonists are disclosed, e.g., in U.S. Pat. Nos. 6,164,282, 6,143,717, 6,140,324 (use of a muscarinic antagonist to treat motion sickness), 6,117,889, 6,103,729, 6,100,046, 6,093,733, 6,066,636 (muscarinic antagonists), 6,060,473, 6,043,255 (muscamic antagonists), 6,037,352, 6,013,766, 6,010,861, 5,994,408, 5,994,330 (topical application of muscarinic agents), 5,977,144, 5,977,138 (ether muscarinic antagonists), 5,958,919, 5,952,349 (muscarinic antagonists for treating memory loss), 5,939,426, 5,935,426, 5,932,481, 5,925,634, 5,922,744, 5,914,349, 5,889,006, 5,883,096, 5,880,159, 5,877,221, 5,877,218, 5,877,173, 5,861,431, 5,852,029, 5,846,819, 5,840,770, 5,837,815, 5,773,458, 5,770,734, 5,756,508, 5,756,501, 5,750,522, 5,739,119, 5,723,494, 5,723,468, 5,716,952 (method for reducing intraocular pressure in the mammalian eye by administration of muscarinic antagonists), 5,712,287, 5,712,271, 5,712,270, 5,712,265 (treatment of glucose metabolism disorders with muscarinic receptor antagonists), 5,710,151, 5,703,209, 5,700,795, 5,693,478, 5,691,323, 5,691,188, 5,683,912, 5,668,155, (treatement of lipid metabolism disorders with muscarinic receptor antagonists), 5,668,144, 5,665,477, 5,652,092, 5,693,913, 5,637,604, 5,629,307, 5,612,351, 5,595,883, 5,594,000, 5,574,044, 5,571,823, 5,567,731, 5,565,475, 5,554,500, 5,534,520, 5,510,478, 5,482,938, 5,480,651, 5,470,856, 5,468,875, 5,464,842, 5,449,522, 5,446,057, 5,441,959, 5,436,128, 5,409,946, 5,407,938, 5,403,585, 5,401,629, 5,385,894, 5,384,243, 5,360,801, 5,356,892, 5,348,955, 5,346,911, 5,344,830, 5,331,002, 5,330,994, 5,324,832, 5,324,729, 5,318,978, 5,312,820, 5,308,846, 5,306,718, 5,292,741, 5,292,726, 5,281,614, 5,278,176, 5,278,968, 5,276,054, 5,264,439, 5,262,178, 5,260,285, 5,258,392, 5,256,667, 5,252,574, 5,250,521, 5,246,944, 5,240,938, 5,238,942, 5,236,942, 5,223,501, 5,221,675, 5,202,328, 5,202,322, 5,200,419, 5,198,438, 5,187,159, 5,183,810, 5,177,095, 5,177,074, 5,175,064, 6,166,206, 6,162,340, 5,162,325, 5,157,040, 5,149,815, 5,128,327, 5,124,335, 5,122,522, 5,100,906, 5,093,333, 5,068,237, 5,066,665, 5,066,663, 5,066,662, 5,055,302, 5,039,601, 4,996,201, 4,988,688, 4,985,560, 4,977,176, 4,977,172, 4,920,102, 4,855,290, 4,835,174, 4,798,841, 4,786,648, 4,665,022, and the like. The entire disclosure of each of these United States patents is hereby incorporated by reference into this specification.

By way of further illustration, dopamine antagonists are suitable candidate antagonists that may be used in the process depicted in FIG. 1. Thus, e.g., one may use one or more of the dopamine antagonists disclosed in U.S. Pat. Nos. 6,203,998 (human dopamine receptor), 6,080,549, 5,883,226, 5,880,260 (dopamine receptor and genes), 5,686,573 (human D5 dopamine receptor protein), 5,594,108, 5,569,601, 5,427,942, 5,422,265, 5,104,858, and the like. The entire disclosure of each of these United States patents is hereby incorporated by reference into this specification.

By way of further illustration, histamine antagonists are suitable candidate antagonists that may be used in the process of this invention. Thus, e.g., one may use one or more of the histamine antagonists described in U.S. Pat. Nos. 6,210,394, 6,136,810, 6,056,715, 5,860,950, 5,820,583, and the like. The entire disclosure of each of these United States patents is hereby incorporated by reference into this specification.

By way of yet further illustration, serotonin antagonists are suitable candidate antagonists that may be used in the process of this invention. Thus, e.g., one may use one or more of the serotonin antagonists described in U.S. Pat. Nos. 6,210,394, 6,180,597, 6,147,109, 6,056,715, 5,877,007, 5,860,950, 5,858,017, 5,820,583, 5,800,385, 5,688,655, 5,266,464, and the like. The entire disclosure of each of these United States patents is hereby incorporated by reference into this specification.

By way of yet further illustration, one may use adrenergic antagonists as the candidate antagonists. Thus, e.g., one may use one or more of the adrenergic antagonists described in U.S. Pat. Nos. 6,203,776, 6,187,756, 6,184,248, 6,077,846, 6,043,224, 5,998,458, 5,610,282, 5,245,011, 5,215,915, and the like. The entire disclosure of each of these United States patents is hereby incorporated by reference into this specification.

It is preferred that the candidate antagonist identified in steps 10 et seq. antagonize functional activity in cholinoceptive cells relevant to cognition preferentially in limbic brain regions (where neuropathology is concentrated) versus neocortical sites. Such antagonist agents might include: muscarinic antagonists that act postsynaptically (i.e., M1, M3 and M5 subtypes) and, moreover, that preferentially antagonize cholinoceptive cells in limbic regions of the brain (i.e., hippocampus, parahippocampal gyrus, cingulate cortex and orbitofrontal cortex) significantly more (i.e., at a ratio of 1.5 or greater) than antagonize neocortical regions (i.e., primary, secondary and tertiary sensory cortices and association areas of the frontal, parietal, temporal and occipital lobes). An example of a candidate drug of this type is (5R,6R)3-propylthio-1,2,5-thiadiazol-4-yl)-1-azabicyclo[3.2.1]octane (PTAC), which is an antagonist at M1, M3 and M4 receptors and a partial agonist at M2 and M4 receptors. This muscarinic antagonism appears to be specific to limbic system neurons insofar as the drug antagonizes electrophysiological responses in limbic-related ventral tegmental dopamine cells (A10), but does not antagonize substantia nigra dopamine cells (A9) as described in Bymaster et al., Eur. J. Pharmacol., 1998, 356:109-119.

Other candidate antagonists which may be utilized in steps 10 et seq. include antipsychotic medicines that are chiefly designed to produce dopamine antagonism but which also exhibit muscarinic antagonism (i.e., clozapine, olanzepine, chlorpromazine, haloperidol).

Because dopamine fibers preferentially innervate hippocampus and limbic cortex as opposed to neocortex, and because their ultimate intracellular effect may have certain similarities to that of acetylcholine, dopamine antagonists without appreciable effects at muscarinic sites may be suitable candidates to combine with anticholinesterases so to block the undesirable effects of the anticholinesterase in the limbic regions. Examples of atypical antipsychotic agents that antagonize dopamine (and also serotonin 5-HT2, histamine, and alpha-l-adrenergic, alpha-2-adrenergic) receptors without appreciable effects on muscarinic receptors include risperidone and quetiapine.

Referring again to FIG. 1, and in step 12, thereof, the candidate antagonists chosen in step 10 are then screened to determine which of them bind to a neurotransmitter receptor which is localized appropriately for phosphorylating MAP-2 in limbic regions.

As is known to those skilled in the art, one may use a radioreceptor assay technique for determining which candidate antagonists bind to a neutrotransmitter receptor which is localized appropriately for phosphorylating MAP-2 in limbic regions. Thus, referring to pages 127-139 of a book edited by H. I. Yamamura et al. entitled “Neurotransmitter Receptor Binding” (Raven Press, New York, 1978), ligand-binding techniques can be used as assay procedures to study brain neurotransmitter receptor sites; the principle of these radioreceptor assay procedures is based on the fact that the amount of radioligand bound to the membrane receptor is quantitatively reduced by the amount of ligand present. Reference may be had, e.g., to U.S. Pat. Nos. 5,202,424, 5,316,754 (in vitro assay of mesangial cell-derived receptors), 5,494,806, 5,556,780, 5,597,920, 5,688,654, 5,840,853, 5,849,708, 5,886,148, and the like. The entire disclosure of each of these United States patents is hereby incorporated by reference into this specification.

By way of further illustration, one may use the screening process described at pages 141-170 of the aforementioned “Neurotransmitter Receptor Binding” book and, in particular, the process described in an Ian Creese article entitled “Receptor Binding as a Primary Drug Screening Device.” As is disclosed at page 166 of this article, “. . . for drugs that exert their effects by interacting directly with neurotransmitter receptors, binding assays provide an inexpensive, rapid, and efficient in vitro screening procedure.” Reference may also be had, e.g., to U.S. Pat. Nos. 6,180,365, 6,180,135, 6,143,521, 6,130,067, 6,127,54, 6,111,091, 6,103,537, 6,103,217, 6,096,868, 6,074,872, 6,071,889, 6,071,779, 6,069,229, 6,043,054, 6,043,052, 6,033,865, 6,031,090, 6,031,003, 6,028,171, 6,022,704, 6,022,696, 6,015,690, 6,011,068, 6,001,581, 5,994,110, 5,985,586, 5,985,214, 5,980,885, 5,968,823, 5,962,314, 5,955,281, 5,942,513, 5,912,326, 5,912,132, 5,910,582, 5,877,173, 5,858,684, 5,854,388, 5,853,999, 5,851,832, 5,849,895, 5,849,734, 5,846,823, 5,837,489, 5,801,232, 5,767,119, 5,766,848, 5,763,569, 5,750,376, 5,744,324, 5,741,651, 5,736,509, 5,707,798, 5,688,938, 6,574,877, 5,639,458, 5,627,168, 5,599,673, 5,565,331, and the like. The entire disclosure of each of these United States patents is hereby incorporated by reference into this specification.

Thus, as is indicated above, binding studies are the appropriate initial screening test for possible candidates. Any prospective agent can be radiolabeled and radiolabeled ligand can be applied to rat brain sections, and in this manner one can obtain a ratio of binding in limbic vs. non-limbic cortical areas. Without wishing to be bound to any particular theory, applicants believe that, in the dopamine system, there are more binding sites for nearly any antagonist in hippocampus and limbic cortex versus neocortex. This kind of difference is not always going to be found for muscarinic antagonists, although at least some compounds must show these differences due to the cytological differences in these areas. These binding studies constitute a preliminary screening process, insofar as such will not always detect functional differences. Even in the absence of binding differences, one might expect cytological differences between limbic and non-limbic cortical regions could underlie a relative difference in strength of the functional response.

Referring again to FIG. 1, and in step 14, the candidate antagonists which pass the initial screening assay of step 12 are then evaluated to determine to what extent, if any, each antagonist has an effect upon the phosphorylation of MAP-2. Reference may be had, e.g., to U.S. Pat. Nos. 6,146,827, 6,030,822, 6,020,142, 6,010,913, 5,994,304, 5,989,907, 5,981,279, 5,861,257, 5,846,780, 5,843,779, 5,821,125, 5,795,735, 5,767,252, 5,580,898, 5,459,036, 5,385,915, and the like. The entire disclosure of each of these United States patents is hereby incorporated by reference into this specification.

By way further illustration, one may use the assay process described in an article by Chiye Oaki and Philip Siekevitz, entitled “Ontogenetic Changes in the Cyclic Adenosine 3′5′-monophosphate-stimulatable Phosphorylation of Cat Visual Cortex Proteins, Particularly of Microtubule-associated Protein 2(MAP 2): effects of Normal and Dark Rearing and of the Exposure to Light,” published in The Journal of Neuroscience, Volume 5, No. 9, pages 2465-2483 (September, 1985). As was disclosed by these authors, biochemical differences were evident in MAP-2 from the visual cortex of a cat that had been dark-reared for 52 days and a litter-mate that had been dark reared and then exposed to light. Autoradiographs were taken, and the dark bands in these autoradiographs indicated proteins that had taken up radioactive phosphate groups during phosphorylation and then were separated according to molecular weight by gel electrophoresis. Proteins from cortical cells were incubated with cyclic AMP and cyclic AMP-dependent protein kinase, together with the radioactive label. Two proteins—synapsin and the kinase—were phosphorylated to the same degree in both cats, but MAP-2 clearly was not. See, e.g., an article appearing by the authors in Scientific American, 1988, Volume 259, pages 56-64.

Referring again to FIG. 1, the step 14 can be used to determine the effect of the candidate antagonist on neocortical cells (such as rat dorsal frontal, parietal, temporal, or occipital cortex tissues), and also on limbic cells (such as rat hippocampus, parahippocampal gyrus, cingulate cortex, and orbital frontal tissues). For each such determination, the extent of the phosphorylation of the MAP-2 may be measured on the “Western blots” by microdensitometry. See, e.g., U.S. Pat. Nos. 6,183,981, 6,162,907, 6,051,393, 5,978,091, 5,966,506, 5,958,909, 5,931,795, 5,910,972, 5,837,467, 5,807,999, 5,780,207, 5,744,287, 5,705,327, 5,670,634, 5,617,213, 5,614,492, and the like. The entire disclosure of each of these United States patents is hereby incorporated by reference into this specification.

By way of further illustration, and as is disclosed in U.S. Pat. No. 5,807,999, “. . . phosphorylated histone Hi can be examined in situ, in cells or tissue samples (e.g., sections mounted on surfaces suitable for microscopic viewing) that have been treated to allow access to nuclear proteins by immunological reagents. In certain embodiments, binding is detected by immunofluorescence, immunohistochemistry, or immunocytochemistry. Alternatively, the binding of a radiolabeled immunoglobulin can be detected by autoradiography.”

Referring again to FIG. 1, in step 16 a comparison is made of the extent to which the antagonist affects the degree of phosphorylation on limbic cells with the extent to which the antagonist affects the degree of phosphorylation on neocortical cells. Thereafter, in step 18 of the process, the ratio of these two steps is calculated.

For an antagonist to be useful in the process of this invention, it is preferred that the ratio of its limbic cell effect to its neocortical cell effect be at least about 1.5. In another embodiment, it is preferred that such ratio be at least about 2.5.

The Dosage of the Antagonist to be Administered

After the particular antagonists which are to be used in the process have been determined in accordance with the procedure of FIG. 1, then one or more of them are administered to a living organism.

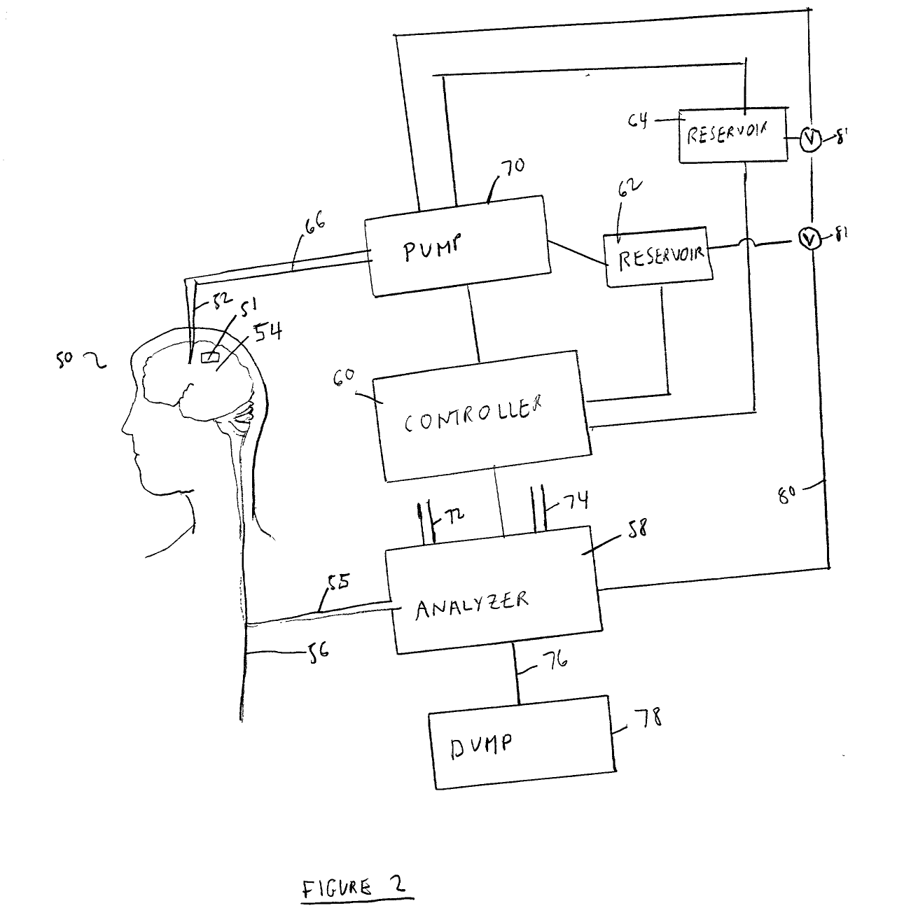

It is preferred that the antagonist be administered to a patient in a dosage of from about 1 to about 300 milligrams per day. The ideal dosage will be that dosage sufficient to maintain the patient in the desired neuroprotective zone, as is described by reference to FIG. 2, which is discussed later in this specification.

In one embodiment, the required dosage of antagonist be administered at least once a day. In another embodiment, the required antagonist is administered two or more times per day.

After a dose of antagonist has been administered to a patient, it is absorbed within the plasma of the patient and thereafter transmitted to the patient's brain cells. The antagonist will reach its maximum concentration in the brain at about the same time as it reaches it peak levels in the plasma.

It is preferred not to administer the anticholinesterease agent to the patient until the antagonist is present in the patient's brain at a specified level, preferably at its peak concentration in the brain of the patient. One can determine by conventional means when the antagonist has reached a specified level (such as its peak level) in the patient's brain.

Thus, by way of illustration and not limitation, one can analyze a patient's cerebrospinal fluid to determine whether particular antagonist (and/or any other analyte, such as an anticholinesterase) has reached its peak level in the brain of a subject. A sample of the cerebrospinal fluid (CSF) may be withdrawn from the patient and analyzed for the presence and concentration of the drug; and this process may be repeated over time until the time it takes for the drug to reach its peak concentration has been determined. Reference may be had, e.g., to U.S. Pat. Nos. 6,184,013, 6,180,604, 5,990,285, 5,981,104, 5,968,547, 5,883,124, 5,852,056, 5,843,994, 5,720,720, 5,690,954, 5,649,904, 5,605,930, 5,552,428, 5,508,039, 5,422,352, 5,213,804, 4,665,086, and the like. The entire disclosure of each of these United States patents is hereby incorporated by reference into this specification.

As will be apparent to those skilled in the art, the same technique may be used to determine the concentration of the anticholinesterase in the patient's blood, of metabolites (or other markers) of the antagonist in the patient's blood, of metabolites (or other markers) of the anticholinesterase in the patient's blood, etc.

In one embodiment, the analyses of the antagonist and anticholinesterase levels in the patient's brain are conducted by one or more implantable sensors.

Process for Selectively Delivering Different Drugs at Different Times

As is indicated in the prior section of this specification, it is preferred to first administer the antagonist to a patient and, thereafter, and only after the antagonist has reached a specified level in the brain of the patient (such as, e.g. its peak concentration in the brain of the patient), deliver the anticholinesterase agent.

One may use prior art devices and processes for delivering the antagonist and the anticholinesterase agent to the brain of the patient at specified concentrations and/or at specified times and/or at different delivery rates.

Thus, by way of illustration and not limitation, one may use the technology described in U.S. Pat. No. 6,010,492, the entire disclosure of which is hereby incorporated by reference into this specification. This patent describes an apparatus for automatic administration of multiple doses of drugs; and such apparatus could be used to deliver the antagonist and the anticholineterase of applicants' process.

By way of further illustration, one may use the technology described in U.S. Pat. No. 6,165,155, the entire disclosure of which is hereby incorporated by reference into this specification. This patent discloses a multi-pathway electronically-controlled drug-delivery system.

Alternatively, or additionally, one may use the technology of U.S. Pat. Nos. 6,057,149 (microscale devices and reactions in microscale devices), 5,417,235 (integrated microvalve structures with monolithic microflow controller), 5,395,626 (multilayered controlled release pharmaceutical dosage form), 6,183,778 (pharmaceutical tablet capable of liberating one or more drugs at different release rates), 6,056,968 (pharmaceutical drug dosage forms providing different release rates), 6,027,748 (coated pharmaceutical tablet), 6,004,582 (multi-layered osmotic device), 5,938,654 (osmotic device for delayed delivery of agent), 5,798,119 (osmotic delivery devices having vapor-permeable coatings), 5,681,583 (multi-layered controlled-release oral solid pharmaceutical forms), 5,662,935 (process for preparing controlled release pharmaceutical forms), 5,549,913 (multilayered matrix systems for the controlled release of active principles), 5,543,913 (diffusion-osmotic controlled drug release composition), 5,391,381 (dispenser capable of delivering a plurality of drug units), 5,945,123 (process for maximizing the effectiveness of substances used to improve health), and the like. The entire disclosure of each of these United States patents is hereby incorporated by reference into this specification.

In one embodiment, the antagonist and the anticholinesterase are administered to the patient, preferably in multiple doses over a 24-hour period of time, such that the peak concentration of the antagonist always precedes in time a peak concentration of the anticholinesterase and, furthermore, that sufficient amounts and doses of the antagonist are delivered so that the concentration of the antagonist never decreases below about 50 percent of its peak concentration. In this embodiment, it is preferred that the ratio of the actual concentration of the antagonist to its peak concentration always exceed the ratio of the actual concentration of the anticholinesterase to its peak concentration.

In another embodiment, the antagonist and the anticholinesterase are administered to the patient, preferably in multiple doses over a 24-hour period of time, such that a preselected, specified value of the antagonist (which may or may not be its peak value) always precedes in time a specified value of the anticholinesterase (which may or may not be its peak value) and, furthermore, that sufficient amounts and doses of the antagonist are delivered so that the concentration of the antagonist never decreases below either a specified value and/or a specified ratio of its concentration to the concentration of the anticholinesterase. As will be apparent, it will often be desirable in such an embodiment to deliver multiple doses of both the antagonist and the anticholinesterase to insure that the patient remains in the desired neuroprotective zone. Thus, e.g., one might first administer a dose of the antagonist, and thereafter administer a dose of the anticholinesterase, and then deliver another dose of the antagonist, and then deliver another dose of the anticholinesterase, etc. It will be apparent that the actual sequence of administrations will depend upon how the patient in question processes the agents being administered to them and, to some extent, the rates of absorption of the agents into the patient's system.

One may use any suitable anticholinesterase in the process of this invention.

As is known to those skilled in the art, acetylcholine is the acetlyated form of choline. Reference may be had, e.g., to U.S. Pat. Nos. 6,211,372, 6,211,364, 6,211,342, 6,211,250, 6,211,245, 6,211,230, 6,211,204, 6,211,182, 6,210,915, 6,210,910, 6,210,394, 6,207,856, 6,207,852, 6,207,836, 6,207,800, 6,207,708, 6,207,681, 6,207,677, 6,207,659, 6,207,410, 6,207,401, 6,207,190, 6,207,160, 6,204,289, 6,204,285, 6,204,264, 6,204,241, 6,104,053, 6,204,022, 6,203,991, 6,201,124, and the like. The entire disclosure of each of these United States patents is hereby incorporated by reference into this specification.

Following neurotransmission, acetylcholine reacts with water to form choline and acetic acid. This hydrolysis reaction is catalyzed by acetylcholinesterase enzyme, which is also often referred to as AChE, true cholinesterase, choline esterase I, or specific cholinesterase. See, e.g., U.S. Pat. Nos. 6,211,141, 6,207,856, 6,207,855, 6,207,853, 6,207,683, 6,207,656, 6,190,723, 6,187,785, 6,184,013, 6,180,597, 6,156,312, 6,150,354, 6,133,276, 6,130,049, 6,130,048, 6,127,410, and the like. The entire disclosure of each of these United States patents is hereby incorporated by reference into this specification.

An “anticholinesterase” is used in the process of this invention. These “anticholinesterases” are discussed, e.g., in U.S. Pat. Nos. 6,211,230, 6,197,788, 6,162,186, 6,150,354, 6,117,454, 6,034,117, 6,025,183 (transgenic animal assay system for anti-cholinesterase substances), 6,024,707, 5,994,330, 5,965,571 (cholinesterase inhibitors for treatment of Parkinson's disease), 5,906,996, 5,886,007 (THA analogs useful as cholinesterase inhibitors), and the like. The entire disclosure of each of these United States patents is hereby incorporated by reference into this specification.

Antichlolinesterases are also known as acetylcholinesterase inhibitors. Reference may be had, e.g., to U.S. Pat. Nos. 6,037,352, 6,037,327, 6,022,683, 5,935,781, 5,886,051, 5,869,484 (phenylcarbamate derivatives suitable as anticholinesterase substances), 5,798,392, 5,789,425 (imidazolidinone derivatives), 5,777,108 (galanthamine derivatives as acetylcholinesterase inhibitors), 5,731,284, 5,589,386 (hydrolysis of cholinesterase inhibitors), 5,585,375, 5,550,253, 5,547,960, 5,541,340, 5,500,188, and the like. The entire disclosure of each of these United States patents is hereby incorporated by reference into this specification.

Anticholinesterases are also known as cholinesterase blockers. Reference may be had, e.g., to U.S. Pat. Nos. 6,150,354, 6,127,370, 6110,742, 6,025,183, 6,022,683, 6,017,929, 6,008,221, 5,990,132, 5,965,571, 5,962,503 (use of cholinesterase inhibitors in the treatment of xerostomia), 5,958,919, 5,932,780, 5,877,173, 5,798,392, 5,756,511, 5,668,117, 5,633,238, 5,589,386 (hydrolysis of cholinesterase inhibitors using parathion hydrolase), 5,576,022 (controlled release tacrine drug system), and the like. The disclosure of each of these United States patents is hereby incorporated by reference into this specification.

Tacrine, also known as tetrahydroaminoacridine (or THA) is a centrally acting, reversible acetylcholine esterase inhibitor; it increases the level of acetylcholine by blocking its degradation. It was approved by the Food and Drug Administration in 1993 under the trade name of “Cognex.” Reference may be had, e.g., to U.S. Pat. Nos. 6,194,403 (tacrine derivatives for treating Alzheimer's disease), 6,124,321 (heteroaryl amines as novel acetyl cholinesterase inhibitors), 6,040,331, 6,027,936, 5,972,376, 5,965,574, 5,948,800, 5,919,778, 5,807,671 (method of screening for genetic predisposition to anticholinesterase therapy), 5,798,392, 5,750,542 (benzisoxazole and benzisothizole derivatives as cholinesterase), 5,171,745, 5,051,410, 5,681,584, 5,668,117, 5,594,002 (benzazabicyclic carbamates as novel cholinesterase inhibitors), 5,576,022 (controlled release tacrine drug delivery system), 5,538,984 (methods of using piperidyl-benisoxazole and benisothiazole derivatives as cholinesterase inhibitors), 5,466,696 (tacrine oxidase inhibitors), 5,428,043 (tricyclic-cyclic amines as novel cholinesterase inhibitors), 5,387,590, 5,004,615, 4,985,256, 4,950,658, 4,895,841 (cyclic amine compounds with activity against acetylcholinesterase), 4,837,164, 4,788,063, and the like. The disclosure of each of these United States patents is hereby incorporated by reference into this specification.

Aricept (also known as Donepezil) is another commercially available acetylcholinesterase inhibitor. See, e.g., U.S. Pat. Nos. 6,197,810, 6,193,993, 6,191,154, 6,166,032, 6,161,044, 6,159,986, 6,156,798, 6,150,354, 6,140,321, 6,124,318, 6,121,046, 6,114,361, 6,110,964, 6,087,392, 6,043,385, 6,037,347, 6,037,327, 6,036,973, 6,034,090, 6,010,702, 5,985,864, 5,981,549, 5,962,535, 5,962,457, 5,958,919, 5,956,125, 5,904,929, 5,900,418, 5,877,173, 5,824,684, 5,760,049, and the like. The entire disclosure of each of these United States patents is hereby incorporated by reference into this specification.

Exelon (also known as rivastigmine) is another commercially available acetylcholinesterase inhibitor. See, e.g., U.S. Pat. Nos. 6,124,318, 6,034,090, 5,904,929, and the like. The entire disclosure of each of these United States patent applications is hereby incorporated by reference into this specification.

In 2001 a review was published regarding the efficacy of “Tacrine for Alzheimer's disease.” The review, which was authored by N. Quzilbash, J. Birks, J. Arrieta Lopez, S. Lewington, and S. Szeto (The Cochrane Library, Issue 1, 2001), “. . . produced no clear results. The results were compatible with tacrine producing improvement, no change or even harm for those with Alzheimer's disease.” The authors stated that “For measure of overall clinical improvement, the intention-to-treat analyses failed to detect any difference between tacrine and placebo . . . Behavioral disturbance, as measured by the Alzheimer's Disease Assessemnt Scalenoncognitive, failed to detect any difference between tacrine and placebo. . . . For cognition function, the effect of tacrine was not statistically significantly different from placebo for the Mini-Mental State Examination score. . . . ”

A Preferred Process of the Invention

FIG. 2 is a schematic diagram of a preferred process for treating a patient 50. As is indicated in FIG. 2, and in the preferred embodiment depicted therein, a catheter 52 is inserted into the brain 54 of patient 50.

One may use any of the catheters commonly used for introducing energy and/or material into a patient's brain. By way of illustration and not limitation, one may use one or more of the devices described in U.S. Pat. Nos. 6,134,460 (spectrophotometers with catheters for measuring internal brain tissue), 6,132,415 (cannula for removing retained fluid and infusing therapeutic fluid, and pump), 6,128,537 (an implantable pump and catheter for infusing drugs into the brain), 6,109,269 (implantable pump and catheter), 5,975,085 (a catheter is used to deliver drugs to a brain to treat schizophrenia), and the like. The entire disclosure of each of these United States patents is hereby incorporated by reference into this specification.

Referring again to FIG. 2, and in the preferred embodiment depicted therein, a catheter 55 is inserted into the spine 56 of the patient to withdraw cerebrospinal fluid therefrom to analyzer 58. This fluid is analyzed in analyzer 58 to determine whether the antagonist and the anticholinesterase are being administered in the proper ratios and at the proper time to provide the desired effects.

When the patient 50 is in the proper neurological zone, there will be decreased levels of phosphorylated tau in the spinal fluid. As is known to those skilled in the art, tau is a microtubule-associated protein. Elevated levels of phosphorylated tau in a patient's brain is a diagnostic marker for Alzheimer's disease. See, e.g., an article by K. Ishiguro et al. entitled “Phosphorylated tau in human cerebrospinal fluid: a diagnostic marker for Alzheimer's disease,” appearing in Neuroscience Letters, 1999 Jul. 30, 270 (2): 91-4. Reference also may be had to U.S. Pat. Nos. 6,194,153, 6,117,978, 6,046,381, 6,020,143, 6,020,139, 5,986,054, 5,840,540, and the like; the entire disclosure of each of these United States patents is hereby incorporated by reference into this specification.

One can establish a baseline level of phosphorylated tau from historical levels of such phosphorylated tau in healthy patients; and a database incorporating such information can be maintained and modified in controller 60. Thereafter, when analyzer 58 determines that the level of phosphorylated tau in the cerebrospinal fluid is at an abnormal level, controller 60 can take corrective measures.

By way of illustration, controller 60 can vary the amount and/or rate of delivery and/or time of delivery of either antagonist (maintained in reservoir 62) and/or anticholinesterase (maintained in reservoir 64) and, as appropriate, feed such materials via line 66 to the patient's brain 68 by means of pump 70. By trying various combinations of drugs and/or delivery times and/or concentrations, the ideal protocol can be determined.

In addition to the historical data indicating an acceptable level of phosphorylated tau in a patient, it will also indicate what combination of delivery conditions for the drugs in question will modify unacceptable levels of phosphorylated tau.

Referring again to FIG. 2, fluid withdrawn via line 55 into analyzer 58 may be treated in such analyzer. Thus, e.g., one or more agents may be added to the fluid within analyzer 58 via line 72. Alternatively, or additionally, one or more forms of electromagnetic energy (such as photonic energy, sound energy, electricity, magnetism) may be fed into analyzer 58 via line 74 and then used to treat the fluid within the analyzer 58.

Fluid within the analyzer 58 which controller 60 decides not to return to the body of patient 50 may be fed via line 76 to dump 78. Fluid which is to be returned to the body of the patient 50 may be fed via line 80 to either reservoir 62 and/or reservoir 64 and/or pump 70. The fluid flow may be controlled, e.g., by valves 82 and 84.

As will be apparent, the system depicted in FIG. 2 will allow one to determine the neuroprotective status of the patient 50 and, when appropriate, takes steps to improve it by either treating the patient's cerebrospinal fluid and/or by varying the amount, time, or rate of delivery of fluid within reservoirs 62 and/or 64.

One may use other means of determining the neurological condition of the patient 50, and/or of administering one or more drugs to such patient.

Thus, e.g., one may use the process of U.S. Pat. 5,869,079, involving the controlled release of a drug by combining hydrophilic and hydrophobic agents to form a biodegradable, sustained release agent. In one embodiment of this patent, a semi-rigid implant for sustained release is provided.

Thus, e.g., one may use the process of U.S. Pat. 5,871,472, the entire disclosure of which is hereby incorporated by reference into this specification. In this process, a static device is implanted, and the device focally releases neuroinhibitory compounds to preselected brain areas.

Thus, e.g., one may use the process of U.S. Pat. No. 5,911,704, the entire disclosure of which is hereby incorporated by reference into this specification. In this process, isolated fetal porcine ventral mesencephalic cells that produce and secrete dopamine are implanted into human brain tissue. As will be apparent, other cells that produce and secrete other agents may be used to practice the process of applicants' invention.

Thus, e.g., one may use the process of U.S. Pat. No. 5,919,802, the entire disclosure of which is hereby incorporated by reference into this specification. In this process, an osmotic minipump is implanted and is caused to deliver 5HT1A antagonist in order to suppress granular cell production. As will be apparent, one may use such an osmotic minipump To deliver other antagonists and/or other agents.

Thus, e.g., one may use the implant described in U.S. Pat. No. 5,965,571, the entire disclosure of which is hereby incorporated by reference into this specification. As is disclosed in this patent, “. . . the cholinesterase inhibitors of the invention can be administered in the form of an implant which compounded with a biodegradable slow-release carrier.” One, thus, may use such an implant in the process of applicants' invention and/or an implant for delivering one or more antagonists.

Thus, e.g., one may use the process described in U.S. Pat. No. 5,978,702, in which epilepsy is treated by brain stimulation and drug infusion and in which a sensor is used to detect a seizure; the entire disclosure of this United States patent is hereby incorporated by reference into this specification.

Thus, e.g., one may use the implantable device of U.S. Pat. 6,007,510, the entire disclosure of which is hereby incorporated by reference into this specification. The device of this patent is adapted to control the flow of fluids within the body.

One may use the device of U.S. Pat. 6,015,572, which contains GDNF secreting cells for treating nerve damage; the entire disclosure of this United States patent is hereby incorporated by reference into this specification. As will be apparent, the device of this patent may be used in applicants' process.

Thus, e.g., one may use the process of U.S. Pat. No. 6,042,579, the entire disclosure of which is hereby incorporated by reference into this specification. The process of this patent treats neurodegenerative disorders by infusing nerve growth factor into the brain. A sensor is used to detect an attribute of the nervous system which reflects the degeneration of nerve cells. A microprocessor algorithm analyzes the output from the sensor in order to regulate the amount of growth factor delivered to the brain. As will be apparent, one may use such sensor and microprocessor algorithm to regulate the balance between the antagonist and the anticholinesterase which is desired in applicants' process.

One may use the process of U.S. Pat. No. 6,056,725, in which indomethacin or nonsteroidal anti-inflammatory agents are delivered directly to the hippocampus or the lateral ventricle through an implanted catheter. Similarly, one can deliver antagonist and/or anticholinesterase directly to the hippocampus or the lateral ventricle with the implanted catheter.

Thus, e.g., one may use the process of U.S. Pat. No. 6,083,523, the entire disclosure of which is hereby incorporated by reference into this specification. In the process of this patent, trophic factors are provided to the proper brain region by implanting a vehicle containing living cells that secrete an appropriate factor.

Thus, e.g., one may use the process described in U.S. Pat. No. 6,094,598, the entire disclosure of which is hereby incorporated by reference into this specification. The process of this patent is used to treat movement disorders by brain stimulation and drug infusion. An implantable signal generator and an implantable pump are used. Additionally, a sensor is used to detect activity resulting from the neural disorder. Thus, e.g., and referring to FIG. 2, sensor 51 may be used, e.g., to electrochemically detect neurotransmitter molecules such as, e.g., dopamine.

Thus, e.g., one may use the process depicted in U.S. Pat. No. 6,101,145, the entire disclosure of which is hereby incorporated by reference into this specification. This patent provides a method and apparatus for detecting and displaying information for an implantable medical device; and it may be used, e.g., in conjunction with sensor 51. In one embodiment, the sensor 51 can be used to monitor acetylcholine levels.

One may use the process described in U.S. Pat. No. 6,123,956, the entire disclosure of which is hereby incorporated by reference into this specification. This patent describes a method for universally distributing therapeutic agents to the brain, utilizing intrathecal administration into the cerebrospinal fluid of a therapeutic agent in an encapsulated form. As will be apparent, the method of this patent can be used to deliver antagonist and/or anticholinesterase agents in an encapsulated form.

Thus, e.g., one may use the oximetric sensor disclosed in U.S. Pat. No. 6,134,474, the entire disclosure of which is hereby incorporated by reference into this specification. This patent discloses spectrophotometers with catheters for measuring internal tissue, and it describes “. . . a . . . oximeter sensor suitable to be worn around the head . . . and capable of responding to tissue oxygen level and blood volume of brain tissue on a real time basis. . . . ”

It is to be understood that the aforementioned description is illustrative and that changes can be made in the apparatus, in the ingredients and their proportions, and in the sequence of combinations and process steps, as well as in other aspects of the invention discussed herein, without departing from the scope of the invention as defined in the following claims.

We claim:

1. A process for treating Alheimer's disease, comprising the steps of administering to a human patient an antagonist of a neurotransmitter receptor which indirectly inhibits phosporylation of microtubule-associated protein-2, and thereafter administering to said patient and anticholinesterase agent, wherein: (a) said antagonist of said neurotransmitter binds to a neurotransmitter receptor which phosphorylates said microtubule-associated protein-2 in limbic cells, (b) said antagonist of said neurotransmitter binds to a neurotransmitter receptor which phosphorylates microtuble-associated protein-2 in neocortical cells, and (c) said antagonist binds to said neurotransmitter receptor in said limbic cells at least 1.5 times as much as it binds to said neurotransmitter receptor in said neocortical cells.

2. The process as recited in claim 1, wherein said antagonist binds to said neurotransmitter receptor in said limbic cells at least 2.5 times as much as it binds to said neurotransmitter receptor in said neocortical cells.

3. The process as recited in claim 2, comprising the steps of administering said antagonist to said patient, allowing said antagonist to reach a specified level in said patient's brain, and thereafter administering said anticholinesterase to said patient.

4. The process as recited in claim 3, wherein said specified level of said antagonist is its peak level.

5. The process as recited in claim 4, further comprising the step of determining the concentrations of said antagonist and said anticholiesterase in said patient's brain.

6. The process as recited in claim 5, further comprising the step of administering an additional amount of said antagonist to said patient after said anticholinesterase has been administered to said patient.

7. The process as recited in claim 6, further comprising the step of administering an additional amount of said anticholinesterase to said patient after said additional amount of said antagonist has been administered to said patient.

8. The process as recited in claim 7, wherein said step of determining the concentrations of said antagonist and said anticholinesterase is conducted by a sensor.

9. The process as recited in claim 8, wherein said sensor is an implantable sensor.

10. A device for treating Alheimer's disease, comprising means for administering to a human patient an antagonist of a neurotransmitter receptor which indirectly inhibits phosporylation of microtubule-associated protein-2, and means for thereafter administering to said patient and anticholinesterase agent, wherein: (a) said antagonist of said neurotransmitter binds to a neurotransmitter receptor which phosphorylates said microtubule-associated protein-2 in limbic cells, (b) said antagonist of said neurotransmitter binds to a neurotransmitter receptor which phosphorylates microtuble-associated protein-2 in neocortical cells, and (c) said antagonist binds to said neurotransmitter receptor in said limbic cells at least 1.5 times as much as it binds to said neurotransmitter receptor in said neocortical cells.

11. The device as recited in claim 10, wherein said device is comprised of a time-release capsule.

12. The device as recited in claim 10, wherein said device is comprised of a time-release tablet.

13. The device as recited in claim 10, wherein said device is comprised of a semi-rigid implant.

14. The device as recited in claim 10, wherein said device is comprised of an implanted pump.

15. The device as recited in claim 10, wherein said device is comprised of a sensor for detecting the concentration of said antagonist in a human brain.

16. The device as recited in claim 15, wherein said sensor for detecting the concentration of antagonist in a human brain is an implantable sensor.

17. The device as recited in claim 16, wherein said device is comprised of a sensor for detecting the concentration of said anticholinesterase within a human brain.

18. The device as recited in claim 17, wherein said sensor for detecting the concentration of said anticholiesterase within said human brain is an implantable sensor.

19. The device as recited in claim 10, wherein said device is comprised of an electronically controlled drug-delivery system.
